(19) United States
(12) Patent Application Publication
(10) Pub. No.: US 2004/0253293 A1
(43) Pub. Date: Dec. 16, 2004

(54) Rate controlled release of a pharmaceutical agent in a biodegradable device

(76) Inventors: Afshin Shafiee, Rochester, NY (US); Joseph C. Salamone, Fairport, NY (US); Dharmendra Jani, Fairport, NY (US); Stephen Paul Bartels, Pittsford, NY (US); Jay F. Kunzler, Canandaigua, NY (US)

Correspondence Address: RITA D. VACCA, BAUSCH & LOMB INCORPORATED, ONE BAUSCH & LOMB PLACE, ROCHESTER, NY, 14604-2701, US

(21) Appl. No.: 10/462,184

(22) Filed: Jun. 16, 2003

(51) Int. Cl. A61F 2/00

(52) U.S. Cl. 424/426

(57) ABSTRACT

Chemical erosion controlled release drug delivery systems are provided that allow controlled release of sustained concentrations of therapeutic agents within a treated area for a prolonged period of time. The favorable solubility characteristics of the chemical erosion controlled release drug delivery systems are controlled through the hydrophobicity and load level of pharmaceutically active agent or drug. Such controlled solubility characteristics allow for manipulation of the drug release rates depending on the particular therapeutic use and the particular needs of the patient.

### FIELD OF THE INVENTION

The present invention relates to a method for controlling the release rate of a pharmaceutical agent from a biodegradable device by controlling the overall hydrophobicity of the biodegradable device. More particularly, the release rate of a pharmaceutical agent from a biodegradable device may be controlled by controlling the load level of a hydrophobic pharmaceutical agent within the biodegradable device.

### BACKGROUND OF THE INVENTION

Conventional drug delivery involving frequent periodic dosing is not ideal or practical in many instances. For example, with more toxic drugs, conventional periodic dosing can result in high initial drug levels at the time of dosing, followed by low drug levels between doses often times below levels of therapeutic value. Likewise, conventional periodic dosing may not be practical or therapeutically effective in certain instances such as with pharmaceutical therapies targeting the inner eye or brain, due to inner eye and brain blood barriers.

During the last two decades, significant advances have been made in the design of controlled release drug delivery systems. Such advances have been made in an attempt to overcome some of the drug delivery shortcomings noted above. In general, controlled release drug delivery systems include both sustained drug delivery systems designed to deliver a drug for a predetermined period of time, and targeted drug delivery systems designed to deliver a drug to a specific area or organ of the body. Sustained and/or targeted controlled release drug delivery systems may vary considerably by mode of drug release within three basic drug controlled release categories. Basic drug controlled release categories include diffusion controlled release, chemical erosion controlled release and solvent activation controlled release. In a diffusion controlled release drug delivery system, a drug is surrounded by an inert barrier and diffuses from an inner reservoir, or a drug is dispersed throughout a non-biodegradable polymer and diffuses from the polymer matrix. In a chemical erosion controlled release drug delivery system, a drug is distributed throughout a biodegradable polymer. The biodegradable polymer is designed to degrade as a result of hydrolysis to then release the drug. In a solvent activation controlled release drug delivery system, a drug is immobilized on polymers within a drug delivery system. Upon solvent activation, the solvent sensitive polymer degrades or swells to release the drug.

The drug release rate from a drug delivery system is typically manipulated through the selection of the biodegradable polymer(s) employed in the system. Biodegradable polymers have varying rates of hydrolytic ability based on the polymers' molecular weights and copolymer ratios, e.g., lactic acid to glycolic acid (LA:GA). The greater the hydrolytic ability of the biodegradable polymer, the greater the drug release rate. The lesser the hydrolytic ability of the biodegradable polymer, the lesser the drug release rate.

Because of the shortcomings of conventional drug delivery noted above, a need exists for methods of controlled release drug delivery systems that allow for manipulation and control of drug release rates depending on the drug to be delivered, the location of delivery, the purpose of delivery and/or the therapeutic requirements of the individual patient.

### SUMMARY OF THE INVENTION

Novel chemical erosion controlled release drug delivery systems of the present invention, produced from one or more biodegradable compositions such as but not limited to 50/50 poly(DL-lactide-co-glycolide) polymer and one or more hydrophobic or hydrophobically-enhanced pharmaceutical agents or drugs, allow for manipulation and control of drug release rates as desired depending on the drug to be delivered, the location of delivery, the purpose of delivery and/or the therapeutic requirements of the individual patient. By varying the hydrophobic or hydrophobically-enhanced pharmaceutical agent drug load within a biodegradable composition, the overall rate of bioerodible degradation of the drug delivery system and hence the drug release rate can be manipulated as desired.

### BRIEF DESCRIPTION OF THE DRAWINGS

FIG. 1 is a graphical representation depicting 100 percent 50/50 poly(DL-lactide-co-glycolide) polymer (PLGA) (placebo) implant hydrolysis absorbance values over time;

FIG. 2 is a graphical representation depicting 100 percent 50/50 PLGA (placebo) implant pH over time;

FIG. 3 is a graphical representation depicting drug release rates over time for 35 percent fluocinolone acetonide (FA) implant—Sample 1;

FIG. 4 is a graphical representation depicting drug release rates over time for 35 percent FA implant—Sample 2;

FIG. 5 is a graphical representation depicting drug release rates over time for 35 percent FA implant—Sample 3;

FIG. 6 is a graphical representation depicting the percent cumulative drug release rates over time for 35 percent FA implant—Sample 1;

FIG. 7 is a graphical representation depicting the percent cumulative drug release rates over time for 35 percent FA implant—Sample 2;

FIG. 8 is a graphical representation depicting the percent cumulative drug release rates over time for 35 percent FA implant—Sample 3;

FIG. 9 is a graphical representation depicting 35 percent FA implant, Samples 1, 2 and 3, pH over time;

FIG. 10 is a graphical representation depicting drug release rates over time for 55 percent FA implant—Sample 1;

FIG. 11 is a graphical representation depicting drug release rates over time for 55 percent FA implant—Sample 2;

FIG. 12 is a graphical representation depicting drug release rates over time for 55 percent FA implant—Sample 3;

FIG. 13 is a graphical representation depicting the percent cumulative drug release rates over time for 55 percent FA implant—Sample 1;

FIG. 14 is a graphical representation depicting the percent cumulative drug release rates over time for 55 percent FA implant—Sample 2;

FIG. 15 is a graphical representation depicting the percent cumulative drug release rates over time for 55 percent FA implant—Sample 3;

FIG. 16 is a graphical representation depicting 55 percent FA implant, Samples 1, 2 and 3, pH over time;

FIG. 17 is a graphical representation depicting 35 percent FA implant, Samples 1, 2 and 3, drug release rates and percent cumulative drug release rates over time;

FIG. 18 is a graphical representation depicting 55 percent FA implant, Samples 1, 2 and 3, drug release rates and percent cumulative drug release rates over time; and

FIG. 19 is a graphical representation depicting 35 percent and 55 percent FA implants, drug release rates and percent cumulative drug release rates over 70 days.

### DETAILED DESCRIPTION OF THE INVENTION

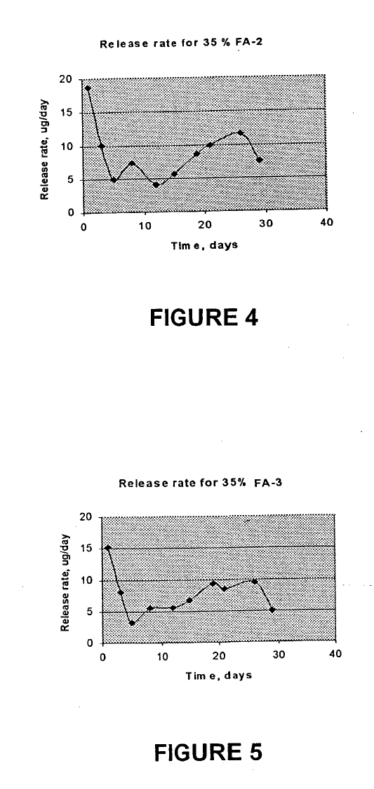

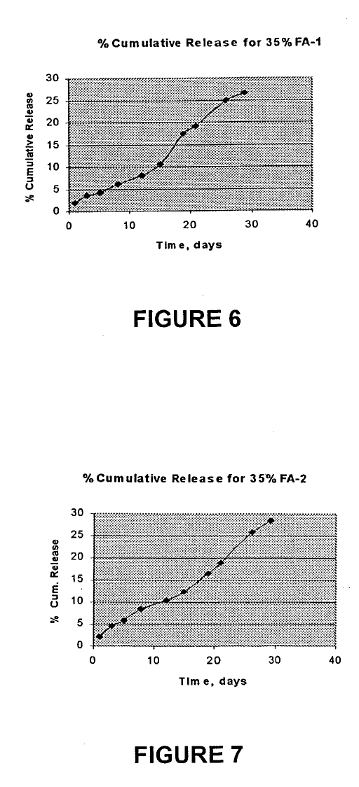

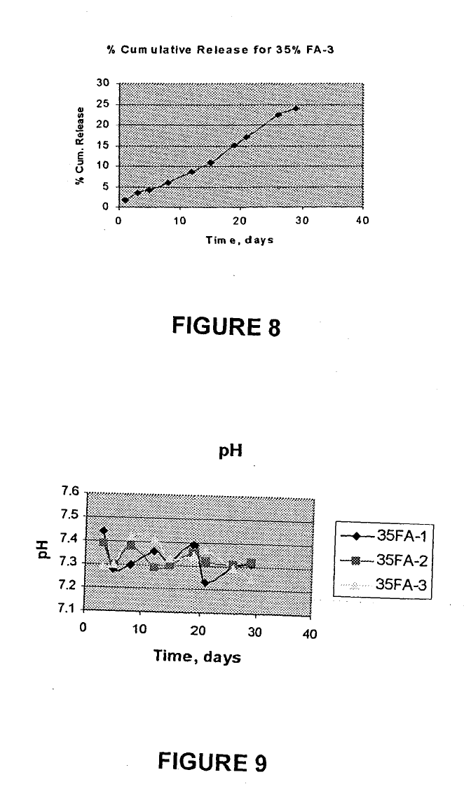

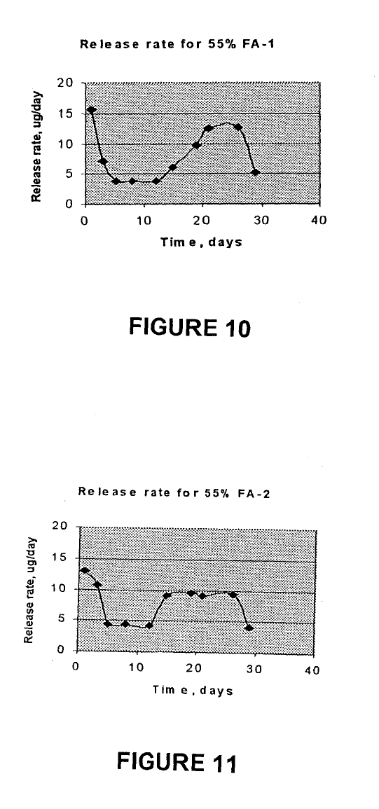

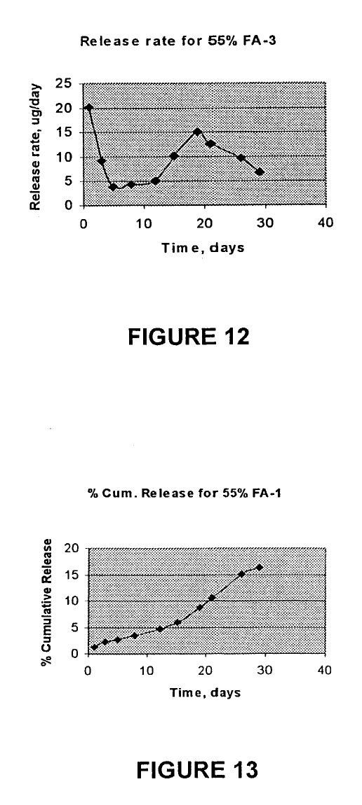

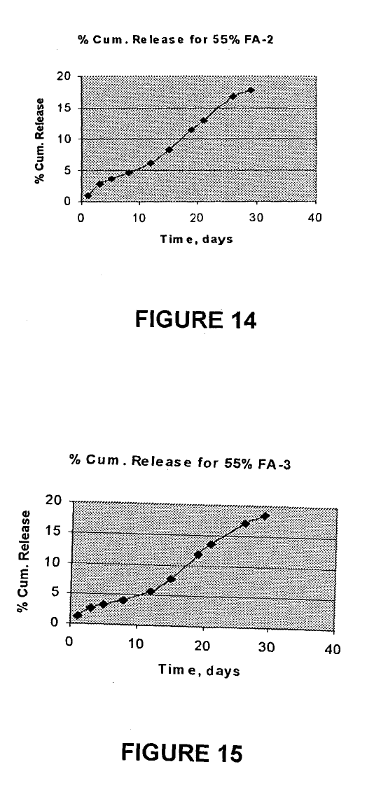

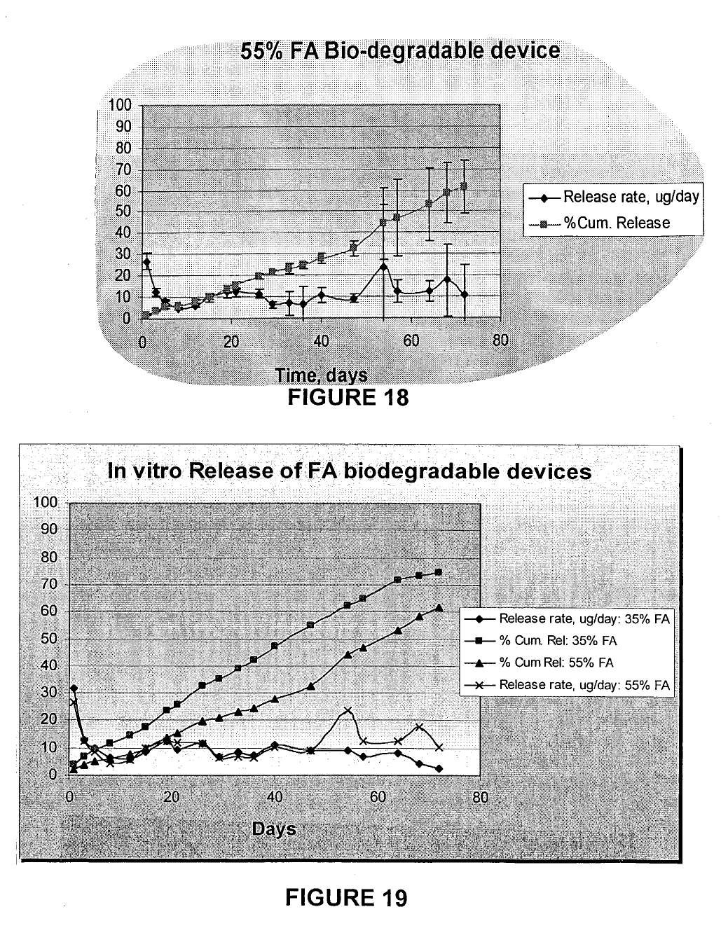

The present invention relates to novel chemical erosion controlled release drug delivery systems, produced from one or more biodegradable compositions such as but not limited to 50/50 poly(DL-lactide-co-glycolide) polymer (PLGA) and one or more hydrophobic or hydrophobically-enhanced pharmaceutical agents or drugs. By varying the hydrophobic or hydrophobically-enhanced pharmaceutical agent or drug load within a biodegradable composition, the overall biodegradable degradation rate of the delivery device and hence the drug release rate can be manipulated as desired. For example, several biodegradable chemical erosion controlled release drug delivery systems were prepared with 35 percent by weight and 55 percent by weight fluocinolone acetonide (FA) loads in 50/50 PLGA through an extrusion process. These drug delivery systems were capable of being inserted through a 0.5 mm diameter cannula used along with the 25-guage needle in the TSV Millenium™ vitrectomy system (Bausch & Lomb Incorporated, Rochester, N.Y.). An in vitro drug release study was conducted to determine the duration and the amount of drug released from the drug delivery systems as illustrated in FIGS. 3-5 and 10-12. Based on a thirty-day study, the 55 weight percent FA systems exhibited slower degradation due to increased hydrophobicity and consequently slower diffusion of the aqueous media resulting in a slower bioerodible degradation. After thirty days, the 35 percent by weight FA systems and the 55 percent by weight FA systems showed a cummulative release of about 25% and 17% respectively, as illustrated in FIGS. 6-8, 13-15, 17 and 18. In both cases, the FA release rate per day was at least approximately 5 μg. After seventy days, the 35 percent by weight FA systems and the 55 percent by weight FA systems showed a cummulative release of about 75% and 61% respectively, as illustrated in FIG. 19. Accordingly, the subject chemical erosion controlled release drug delivery systems allow for control of drug release rates based on the load of the hydrophobic or hydrophobically-enhanced drug to be delivered.

For purposes of the present invention, suitable biodegradable polymers for use in the subject chemical erosion controlled release drug delivery systems include for example but are not limited to poly(lactide)s, poly(glycolide)s, poly(lactide-co-glycolide)s, poly(lactic acid)s, poly(glycolic acid)s, poly(lactic acid-co-glycolic acid)s, polycaprolactones, polycarbonates, poly(ester amide)s, polyanhydrides, poly(amino acid)s, polyorthoesters, polyacetals, polycyanoacrylates, poly(ether ester)s, polydioxanones, poly(alkylene alkylate)s, copolymers of polyethylene glycol and polyorthoester, biodegradable polyurethanes, and blends and copolymers thereof.

For purposes of the present invention, suitable hydrophobic pharmaceutical agents or drugs for use in the subject chemical erosion controlled release drug delivery systems include any pharmaceutical agents or drugs that are hydrophobic, as defined herein as meaning sparingly soluble or slightly soluble in water, i.e., less than one percent drug/solution. Likewise, hydrophilic drugs or drugs having low hydrophobicity can be used in accordance with the present invention by increasing the hydrophobicity thereof. Such hydrophobicity-enhanced drugs are produced by admixing the hydrophilic drug or drug having low hydrophobicity with a suitable biocompatible hydrophobic agent. Suitable biocompatible hydrophobic agents include for example but are not limited to glycerol triacetate, glycerol diacetate, diethyl phthalate, dimethyl phthalate, phthalate esters, phosphate esters, fatty acid esters, glycerol derivatives, acetyl triethyl citrate, dibutyl tartrate and combinations thereof. Such hydrophobic agents influence drug release rate by filling the matrix polymer interstices. By filling the matrix polymer interstices, hydrophobic agents impede water diffusion into the bulk of the drug delivery system both by their hydrophobicity and by serving as physical blockages. Through the impediment of water diffusion, the hydrolytic degradation rate of the drug delivery system is reduced.

Suitable hydrophobic drugs, or drugs suitable upon hydrophobicity enhancement for use in the present invention include for example but are not limited to ametantrone, amphotericin B, annamycin, cyclosporin, daunorubicin, diazepam, doxorubicin, elliptinium, etoposide, fluocinolone acetonide, ketoconazole, methotrexate, miconazole, mitoxantrone, nystatin, phenytoin and vincristine. Other suitable pharmaceutically active agents include but are not limited to cytokines and steroidal hormones for example estragenic, e.g., estradiol, and androgenic, e.g., testosterone, hormones, or other hormones that comprise a sterol backbone. Mixtures of more than one drug can also be incorporated into one drug delivery system for the purpose of co-administration.

Other pharmaceutically active agents or drugs useful in the chemical erosion controlled release drug delivery system of the present invention include for example but are not limited to anti-glaucoma agents such as for example but not limited to intraocular pressure lowering agents such as for example diamox, neuroprotection agents such as for example nimodipine, beta blockers such as for example timolol maleate, betaxolol and metipranolol, mitotics such as for example pilocarpine, acetylcholine chloride, isofluorophate, demacarium bromide, echothiophateiodide, phospholine iodide, carbachol and physostigimine, epinephrine and salts such as for example dipivefrin hydrochloride, dichlorphenamide, acetazolamide and methazolamide; anti-diabetic edema agents such as for example but not limited to steroids such as for example fluocinolone, and anti-vascular endothelial growth factors (VEGF) receptors such as for example VEGF receptor tyrosine kinase inhibitors, pyrrolyl-methylene-indolinones and C₆₋₄₅ phenyl amino alkoxy quinazolines; anti-proliferative vitreoretinopathy agents such as for example but not limited to fluocinolone acetonide, dexamethasone, prednisolone and triamcinolone acetonide; anti-inflammatory agents such as for example but not limited to steroids such as for example hydrocortisone, hydrocortisone acetate, dexamethasone, fluocinolone, medrysone, methylprednisolone, prednisolone, prednisolone acetate, fluoromethalone, betamethasone and triamcinolone acetonide and immunological response modifiers such as for example cyclosporin; anti-ocular angiogenesis agents such as for example but not limited to anti VEGF receptors such as for example VEGF receptor tyrosine kinase inhibitors, pyrrolyl-methylene-indolinones and C₆₋₄₅ phenyl amino alkoxy quinazolines, anti-mobility agents such as for example cytochalasin B, steroids such as for example fluocinolone acetonide dexamethasone and prednisolone, matrix metalloproteinase (MMP) inhibitors such as for example benzodiazepine sulfonamide hydroxamic acids, and humanized antibodies, aptamers and peptides that are formulated to become sparingly soluble; antibiotics such as for example but not limited to ganciclovir; angiogenesis targeting agents such as for example but not limited to angiogenic growth factors such as for example VEGF, VEGF receptors, integrins, tissue factors, prostaglandin-cyclooxygenase 2 and MMPs; anti-cataract and anti-diabetic retinopathy agents such as for example but not limited to the aldose reductase inhibitors, tolrestat, lisinopril, enalapril and statil, thiol cross-linking agents, anticancer agents such as for example but not limited to retinoic acid, methotrexate, adriamycin, bleomycin, triamcinolone, mitomycin, cisplatinum, vincristine, vinblastine, actinomycin-D, ara-c, bisantrene, activated cytoxan, melphalan, mithramycin, procarbazine and tamoxifen, immune modulators, anti-clotting agents such as for example but not limited to tissue plasminogen activator, urokinase and streptokinase, anti-tissue damage agents such as for example but not limited to superoxide dismutase, proteins and nucleic acids such as for example but not limited to mono- and poly-clonal antibodies, enzymes, protein hormones and genes, gene fragments and plasmids, steroids, particularly anti-inflammatory or anti-fibrous agents such as for example but not limited to lodeprednol, etabonate, cortisone, hydrocortisone, prednisolone, prednisome, dexamethasone, progesterone-like compounds, medrysone (HMS) and fluorometholone, non-steroidal anti-inflammatory agents such as for example but not limited to ketrolac tromethamine, dichlofenac sodium and suprofen, antibiotics such as for example but not limited to loridine (cephaloridine), chloramphenicol, clindamycin, amikacin, tobramycin, methicillin, lincomycin, oxycillin, penicillin, amphotericin B, polymyxin B, cephalosporin family, ampicillin, bacitracin, carbenicillin, cepholothin, colistin, erythromycin, streptomycin, neomycin, sulfacetamide, vancomycin, silver nitrate, sulfisoxazole diolamine and tetracycline, other antipathogens including anti-viral agents such as for example but not limited to idoxuridine, trifluorouridine, vidarabine (adenine arabinoside), acyclovir (acycloguanosine), pyrimethamine, trisulfapyrimidine-2, clindamycin, nystatin, flucytosine, natamycin, and miconazole, piperazine derivatives such as for example but not limited to diethylcarbamazine, and cycloplegic and mydriatic agents such as for example but not limited to atropine, cyclogel, scopolamine, homatropine and mydriacyl.

Other suitable pharmaceutically active agents or drugs include anticholinergics, anticoagulants, antifibrinolytics, antihistamines, antimalarials, antitoxins, chelating agents, hormones, immunosuppressives, thrombolytics, vitamins, salts, desensitizers, prostaglandins, amino acids, metabolites and antiallergenics.

Pharmaceutical agents or drugs of particular interest include hydrocortisone (5-20 mcg/l as plasma level), gentamycin (6-10 mcg/ml in serum), 5-fluorouracil (˜30 mg/kg body weight in serum), sorbinil, interleukin-2, phakan-a (a component of glutathione), thioloa-thiopronin, bendazac, acetylsalicylic acid, trifluorothymidine, interferon (α, β and γ), immune modulators such as for example but not limited to lymphokines and monokines and growth factors.

The drug hydrophobicity and load size within the drug delivery system dictates the rate of bioerodible degradation, and is a primary factor controlling the rate of drug release. Thus, by controlling the hydrophobicity of the drug and the drug load size within the drug delivery system, particular characteristics or properties are achieved. The particular characteristics or properties achieved may then be manipulated to achieve the desired rate of drug release. The desired rate of drug release may be determined based on the drug to be delivered, the location of delivery, the purpose of delivery and/or the therapeutic requirements of the individual patient.

The chemical erosion controlled release drug delivery systems of the present invention are described in still greater detail in the examples that follow.

### EXAMPLE 1

### Chemical Erosion Controlled Release Drug Delivery System Sample Preparation and Study

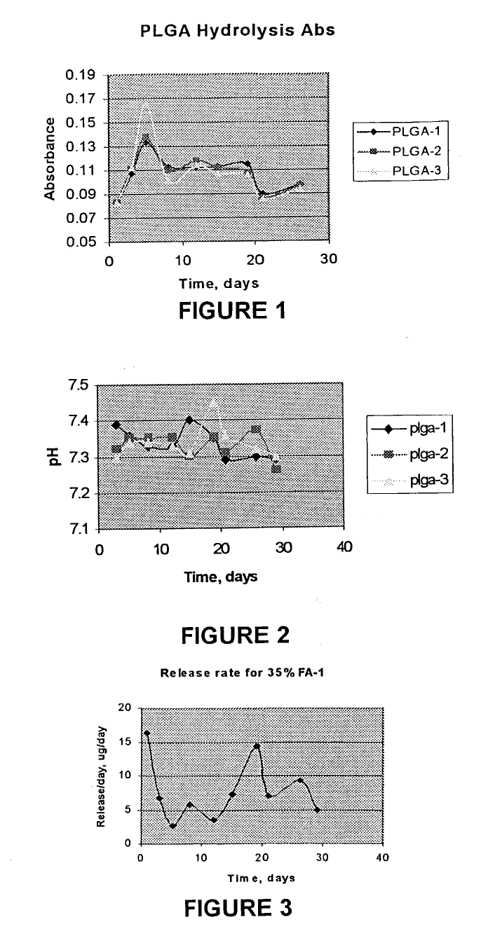

An Atlas™ lab mixing extruder (LME) (Dynisco Instruments, Franklin, Mass.) was used to mix and extrude PLGA/FA strands at 35 percent and 55 percent loadings and PLGA placebo filaments, each approximately 0.5 mm in diameter. These cylindrical filaments were stored in a dessicator unit. Three samples per loading approximately 0.5 mm diameter and 1 cm in length were cut, weighed and placed individually in a centrifuge tube containing 50 ml phosphate buffered solution, pH=7.4. Each sample was allowed to adhere to the wall of the centrifuge tube and placed on a rotating mixer at 8 revolutions per minute (rpm). All samples were then placed in an oven at 37° C. At periodic intervals, 15 ml solution samples from the 50 ml reservoir were removed and replaced with equal volume of fresh phosphate buffered saline (PBS). The pH of the solution samples was measured. The solution samples were then diluted with 15 ml of fresh PBS and mixed thoroughly. The absorbance values were read on a UV/VIS spectrophotometer and peak values corresponding to glycolic acid and FA were read for each sample period as illustrated in FIG. 1. The release rate per day and percent cummulative release were determined.

50/50 DL-PLGA is an amorphous polymer. The primary pathway for PLGA biodegradation is through water diffusion into the polymer matrix, random hydrolysis, matrix fragmentation followed by extensive hydrolysis along with phagocytosis, diffusion and metabolism. For the first 30 days of the study, a transparent PLGA sample showed signs of increasing water diffusion as evidenced by the change in refractive index of the implant. No macro-fragmentation was visible. Other factors affecting the hydrolysis and consequently drug release are the surface area of the implant, polymer crystallinity and hydrophilicity as well as pH and temperature of the surrounding media. Extrusion of the polymer induces crystallinity which slows down degradation relative to other modes of fabrication such as compression molding or, to a lesser extent, injection molding. Molecular weight and glycolide content in the copolymer can also significantly affect the rate of hydrolysis as well as the mixing speed, rpm, of the tube tumbler. Peak absorbance values for glycolic acid show a relatively stable hydrolysis after an initial peak produced from surface diffusion. The system showed adequate buffering as seen by the narrow pH range measured over 30 days, as illustrated in FIG. 2.

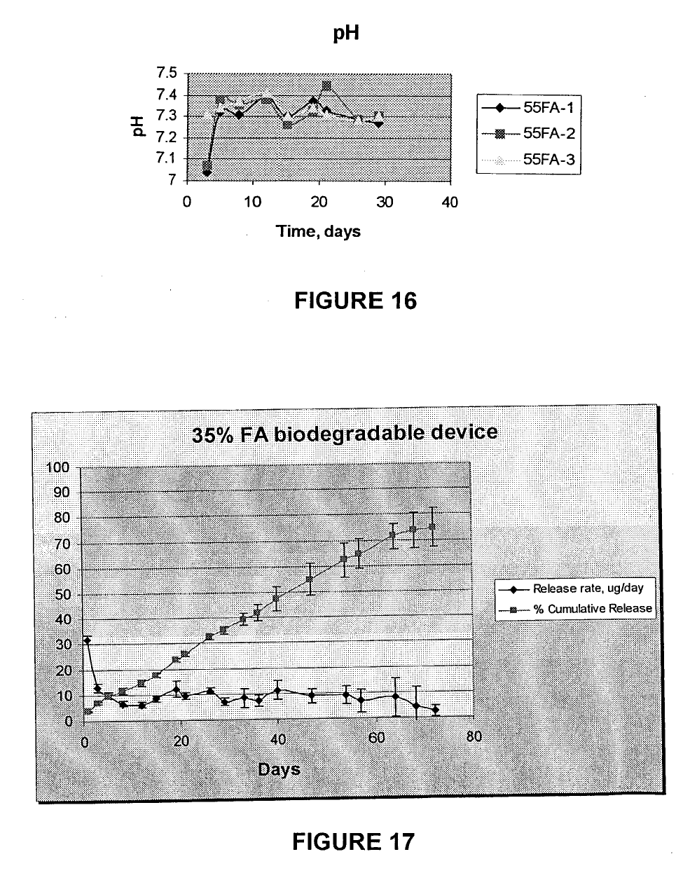

Presence of a hydrophobic compound, fluocinolone acetonide in PLGA significantly slows down the water diffusion rate as evidenced by the relatively smaller change in the size of the implant. The surface of the implant also appeared to be smoother than the PLGA implant. For the most part, the FA release rate exceeded 5 μg/day with a cumulative release of about 25 percent of the approximately 850 μg FA present in the implant. The system pH showed little change over the course of the 30 days, as illustrated in FIGS. 9 and 16, influenced by the slower PLGA hydrolysis and low acid constant, kₐ, for FA.

The 55 percent FA implants seem to be releasing at roughly the same rate as the 35 percent implant. The samples also appeared to be holding intact at the same level as the 35 percent implants. The pH of the system seems to be well buffered as well.

In conclusion, similar release rates per day were observed for both 35 percent and 55 percent FA implants during the first 30 days of study which seems to be primarily a diffusion controlled process. The percent cumulative release of FA, based on estimated FA loading, observed so far is significantly less for the 55 percent implants relative to the 35 percent implants.

Chemical erosion controlled release drug delivery systems of the present invention may be manufactured in any shape or size suitable for the intended purpose for which they are intended to be used. For example, for use as an inner back of the eye implant, the subject chemical erosion controlled release drug delivery system would preferably be no larger in size than 3 mm². Methods of manufacturing the subject chemical erosion controlled release drug delivery systems includes cast molding, extrusion, and like methods known to those skilled in the art. Once manufactured, the subject chemical erosion controlled release drug delivery systems are packaged and sterilized using customary methods known to those skilled in the art.

Chemical erosion controlled release drug delivery systems of the present invention may be used in a broad range of therapeutic applications. In the field of ophthalmology for example, the subject controlled release drug delivery system is used by implantation within the interior portion of an eye. However, the subject chemical erosion controlled release drug delivery system may likewise be used in accordance with other surgical procedures known to those skilled in the field of ophthalmology.

While there is shown and described herein chemical erosion controlled release drug delivery systems and methods of making and using the same, it will be manifest to those skilled in the art that various modifications may be made without departing from the spirit and scope of the underlying inventive concept. The present invention is likewise not intended to be limited to particular monomers, copolymers and systems described herein except insofar as indicated by the scope of the appended claims.

We claim:

1. A chemical erosion controlled release drug delivery system comprising:
a biodegradable polymer with a therapeutically effective amount of at least one hydrophobic or hydrophobically-enhanced pharmaceutically active agent with rate of chemical erosion and release rate of said active agent controlled by said active agent.

2. A chemical erosion controlled release drug delivery system comprising:
a biodegradable polymer with a therapeutically effective amount of at least one hydrophobic or hydrophobically-enhanced pharmaceutically active agent present in an amount sufficient to control rate of said active agent release from said biodegradable polymer.

3. The drug delivery system of claim 1 or 2 wherein said biodegradable polymer is selected from the group consisting of poly(lactide)s, poly(glycolide)s, poly(lactide-co-glycolide)s, poly(lactic acid)s, poly(lactic acid-co-glycolic acid)s, polycaprolactones, polycarbonates, poly(ester amide)s, polyanhydrides, poly(amino acid)s, polyorthoesters, polyacetals, polycyanoacrylates, poly(ether ester)s, polydioxanones, poly(alkylene alkylate)s, copolymers of poly(ethylene glycol) and polyorthoesters, biodegradable polyurethanes and blends and copolymers thereof.

4. The drug delivery system of claim 1 or 2 wherein said hydrophobically-enhanced pharmaceutically active agents are produced by admixing a hydrophilic pharmaceutically active agent or a pharmaceutically active agent of low hydrophobicity with a hydrophobic agent.

5. A method of producing a hydrophobically-enhanced pharmaceutically active agent comprising:
admixing a hydrophilic pharmaceutically active agent or a pharmaceutically active agent of low hydrophobicity with a hydrophobic agent.

6. The drug delivery system of claim 4 wherein said hydrophobic agent is selected from the group consisting of glycerol triacetate, glycerol diacetate, diethyl phthalate, dimethyl phthalate, phthalate esters, phosphate esters, fatty acid esters, glycerol derivatives, acetyl triethyl citrate, dibutyl tartrate and combinations thereof.

7. The method of claim 5 wherein said hydrophobic agent is selected from the group consisting of glycerol triacetate, glycerol diacetate, diethyl phthalate, dimethyl phthalate, phthalate esters, phosphate esters, fatty acid esters, glycerol derivatives, acetyl triethyl citrate, dibutyl tartrate and combinations thereof.

8. The drug delivery system of claim 1 or 2 wherein said at least one pharmaceutically active agent is selected from the group consisting of ametantrone, amphotericin B, annamycin, cyclosporin, daunorubicin, diazepam, doxorubicin, elliptinium, etoposide, fluocinolone acetonide, ketoconazole, methotrexate, miconazole, mitoxantrone, nystatin, phenytoin, lodeprednol, triamcinolone acetonide and vincristine.

9. The drug delivery system of claim 1 or 2 wherein said at least one pharmaceutically active agent is selected from the group consisting of cytokines and steroidal hormones.

10. The drug delivery system of claim 1 or 2 wherein said at least one pharmaceutically active agent is selected from the group consisting of anti-glaucoma agents, neuroprotection agents, beta blockers, mitotics, epinephrine, anti-diabetic edema agents, anti-vascular endothelial growth factors (VEGF) receptors, pyrrolyl-methylene-indolinones, C₆₋₄₅ phenyl amino alkoxy quinazolines, anti-proliferative vitreoretinopathy agents, anti-inflammatory agents, immunological response modifiers, anti-ocular angiogenesis agents, anti-mobility agents, steroids, matrix metalloproteinase (MMP) inhibitors, humanized antibodies, aptamers, peptides, antibiotics, angiogenesis targeting agents, anti-cataract and anti-diabetic retinopathy agents, thiol cross-linking agents, anticancer agents, immune modulators, anti-clotting agents, anti-tissue damage agents, proteins, nucleic acids, anti-fibrous agents, non-steroidal anti-inflammatory agents, antibiotics, antipathogens, piperazine derivatives, cycloplegic and mydriatic agents anticholinergics, anticoagulants, antifibrinolytics, antihistamines, antimalarials, antitoxins, chelating agents, hormones, immunosuppressives, thrombolytics, vitamins, salts, desensitizers, prostaglandins, amino acids, metabolites and antiallergenics.

11. The drug delivery system of claim 1 or 2 wherein said at least one pharmaceutically active agent is selected from the group consisting of hydrocortisone, gentamycin, 5-fluorouracil, sorbinil, interleukin-2, phakan-a, thioloa-thiopronin, bendazac, acetylsalicylic acid, trifluorothymidine, interferon, immune modulators and growth factors.

12. A method of making the drug delivery system of claim 1 or 2 comprising:
encapsulating in a biodegradable polymer a therapeutically effective amount of at least one pharmaceutically active agent.

13. The method of claim 12 wherein said biodegradable polymer is selected from the group consisting of poly(lactide)s, poly(glycolide)s, poly(lactide-co-glycolide)s, poly(lactic acid)s, poly(lactic acid-co-glycolic acid)s, polycaprolactones, polycarbonates, poly(ester amide)s, polyanhydrides, poly(amino acid)s, polyorthoesters, polyacetals, polycyanoacrylates, poly(ether ester)s, polydioxanones, poly(alkylene alkylate)s, copolymers of polyethylene glycol and polyorthoester, biodegradable polyurethanes and blends and copolymers thereof.

14. The method of claim 12 wherein said at least one pharmaceutically active agent is selected from the group consisting of ametantrone, amphotericin B, annamycin, cyclosporin, daunorubicin, diazepam, doxorubicin, elliptinium, etoposide, fluocinolone acetonide, ketoconazole, methotrexate, miconazole, mitoxantrone, nystatin, phenytoin, lodeprednol, triamcinolone acetonide and vincristine.

15. The method of claim 12 wherein said at least one pharmaceutically active agent is selected from the group consisting of cytokines and steroidal hormones.

16. The method of claim 12 wherein said at least one pharmaceutically active agent is selected from the group consisting of anti-glaucoma agents, neuroprotection agents, beta blockers, mitotics, epinephrine, anti-diabetic edema agents, anti-vascular endothelial growth factors (VEGF) receptors, pyrrolyl-methylene-indolinones, C₆₋₄₅ phenyl amino alkoxy quinazolines, anti-proliferative vitreoretinopathy agents, anti-inflammatory agents, immunological response modifiers, anti-ocular angiogenesis agents, anti-mobility agents, steroids, matrix metalloproteinase (MMP) inhibitors, humanized antibodies, aptamers, peptides, antibiotics, angiogenesis targeting agents, anti-cataract and anti-diabetic retinopathy agents, thiol cross-linking agents, anticancer agents, immune modulators, anti-clotting agents, anti-tissue damage agents, proteins, nucleic acids, anti-fibrous agents, non-steroidal anti-inflammatory agents, antibiotics, antipathogens, piperazine derivatives, cycloplegic and mydriatic agents anticholinergics, anticoagulants, antifibrinolytics, antihistamines, antimalarials, antitoxins, chelating agents, hormones, immunosuppressives, thrombolytics, vitamins, salts, desensitizers, prostaglandins, amino acids, metabolites and antiallergenics.

17. The method of claim 12 wherein said at least one pharmaceutically active agent is selected from the group consisting of hydrocortisone, gentamycin, 5-fluorouracil, sorbinil, interleukin-2, phakan-a, thioloa-thiopronin, bendazac, acetylsalicylic acid, trifluorothymidine, interferon, immune modulators and growth factors.

18. A method of using the drug delivery system of claim 1 or 2 comprising:
creating an incision within an eye; and
implanting said drug delivery system within said eye through said incision.

19. A method of using the drug delivery system of claim 1 or 2 comprising:
creating an incision within an eye; and
implanting said drug delivery system within said eye through said incision using a cannula used along with a needle of a vitrectomy system.

20. A method of using a drug delivery system comprising: creating an incision within an eye; and
implanting said drug delivery system within said eye through said incision using a cannula used along with a needle of a vitrectomy system.
